(19) United States
(12) Patent Application Publication
(10) Pub. No.: US 2025/0256091 A1
(43) Pub. Date: Aug. 14, 2025

(54) MASSAGE SUIT SYSTEM

(71) Applicant: GENAVIEVE BOYLES, BENBROOK, TX (US)

(72) Inventor: GENAVIEVE BOYLES, BENBROOK, TX (US)

(21) Appl. No.: 18/436,255

(22) Filed: Feb. 8, 2024

(51) Int. Cl. A61N 1/04; A41D 1/00; A61N 1/36

(52) U.S. Cl. CPC A61N 1/0484; A41D 1/002; A61N 1/3603

(57) ABSTRACT

A massage suit system includes a wearable garment and an operating unit coupled thereto. The operating unit is configured to include a power supply and a control unit. The operating unit is coupled to a first side of the wearable garment. A pair of conductive patches are coupled to a second side of the wearable garment and are in communication with a pair of electrodes. The electrodes are in wired communication with the operating unit and are configured to selectively provide electrical current to the conductive patches. An electronic device is configured to communicate with the operating unit to permit a user to regulate activation of the electrodes. The system is configured to be used in both static and dynamic motion environments wherein the user may be resting or moving. A portable cleaning case is also provided.

### BACKGROUND OF THE INVENTION

### 1. Field of the Invention

The present application relates to a massage suit, and more particularly to a wearable garment containing a plurality of e-stim units to provide localized treatment.

### 2. Description of Related Art

E-stim is a medical device that is used to help accelerate recovery from an injury and provide relief from painful or uncomfortable symptoms. An example may be that of an injury or stroke, or dealing with pain from fibromyalgia or other conditions. E-stim is designed to send mild electrical pulses through the skin to help stimulate injured muscles or manipulate nerves to reduce pain. An objective is to mimic the action of signals coming from neurons in the nervous system to target muscles or nerves. By repeating muscle contraction and relaxation, the muscles may gain strength and be trained to respond to the body's natural signals. Where focus is on the nerves, an e-stim sends signals on a different wavelength so they reach the nerves and not the muscles. The idea there is to block pain receptors from being sent from nerves to the brain.

Two main types of e-stim are transcutaneous electrical nerve stimulation (TENS) and electrical muscle stimulation (EMS). E-stim devices are typically handheld or small devices applied to the selected body area by either a medical professional or a user. The device uses small electrode pads that are stuck to the body to provide the electrical stimulation desired. Removal of the pads can cause discomfort as they are attached with an adhesive.

A common disadvantage of present systems is that they often require the user to be stationary and only treat one area of the body at a single time. Although strides have been made, shortcomings remain.

### BRIEF SUMMARY OF THE INVENTION

It is an object of the present application to provide a massage suit comprising wearable garments with incorporated e-stim devices to enable treatment of a user while on the go, both when non-stationary and when stationary. The wearable garments may include sleeves, shirts, shorts, socks, and other garments. The system is configured to selectively regulate operation of e-stim devices in the wearable garments as a whole treatment or individually.

It is a further object of the present application that permit the user/wearer to receive treatment indiscreetly worn under the clothing and while moving about. Furthermore, the user may regulate operation of the massage suit via wireless communications with an electronic device. Each item of clothing may be regulated either individually or collectively as a whole. Another object of the present system is to provide a portable cleaning case configured to safely clean the wearable garments. Furthermore, an object of the present system may be to permit adult stimulation to selected parts of the body.

Ultimately the invention may take many embodiments. In these ways, the present invention overcomes the disadvantages inherent in the prior art. The more important features have thus been outlined in order that the more detailed description that follows may be better understood and to ensure that the present contribution to the art is appreciated. Additional features will be described hereinafter and will form the subject matter of the claims that follow.

Many objects of the present application will appear from the following description and appended claims, reference being made to the accompanying drawings forming a part of this specification wherein like reference characters designate corresponding parts in the several views.

Before explaining at least one embodiment of the present invention in detail, it is to be understood that the embodiments are not limited in its application to the details of construction and the arrangements of the components set forth in the following description or illustrated in the drawings. The embodiments are capable of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the various purposes of the present design. It is important, therefore, that the claims be regarded as including such equivalent constructions insofar as they do not depart from the spirit and scope of the present application.

### BRIEF DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a view of a massage suit system according to an embodiment of the present application.

FIG. 2 is an enlarged side view of an e-stim patch located within the massage suit garment of FIG. 1.

FIG. 3 is a representative front view of the massage suit system of FIGS. 1-2.

FIG. 4 is a representative rear view of the massage suit system of FIGS. 3-4.

FIG. 5-10 are tables of e-stim locations and associated data in the massage suit system of FIGS. 1-2.

FIG. 11 is a perspective view of a portable cleaning case in the massage suit system of FIGS. 1-2.

While the embodiments and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE INVENTION

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the embodiments described herein may be oriented in any desired direction.

The embodiments and method in accordance with the present application overcomes one or more of the above-discussed problems commonly associated with the prior art discussed previously. In particular, the massage suit system of the present application is configured to include wearable garments having an operating unit capable of communicating with software on an electronic device. Each wearable garment includes an operating unit in wired communication with one or more electrodes. The electrodes are configured to provide e-stim treatments to selected muscular areas of the user's body. The electronic device includes application software configured to communicate with the operating units located on each wearable garment so as to regulate the treatment at each electrode. These and other unique features are discussed below and illustrated in the accompanying drawings.

The embodiments and method will be understood, both as to its structure and operation, from the accompanying drawings, taken in conjunction with the accompanying description. Several embodiments of the assembly may be presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

The embodiments and method of the present application is illustrated in the associated drawings. The massage suit system includes a wearable garment and an operating unit coupled thereto. The operating unit is configured to include a power supply and a control unit. The operating unit is coupled to a first side of the wearable garment. A pair of conductive patches are coupled to a second side of the wearable garment and are in communication with a pair of electrodes. The electrodes are in wired communication with the operating unit and are configured to selectively provide electrical current to the conductive patches. An electronic device is configured to communicate with the operating unit to permit a user to regulate activation of the electrodes. The system is configured to be used in both static and dynamic motion environments wherein the user may be resting or moving. Additional features and functions are illustrated and discussed below.

Referring now to the Figures wherein like reference characters identify corresponding or similar elements in form and function throughout the several views. The following Figures describe embodiments of the present application and its associated features. With reference now to the Figures, embodiments of the present application are herein described. It should be noted that the articles “a”, “an”, and “the”, as used in this specification, include plural referents unless the content clearly dictates otherwise.

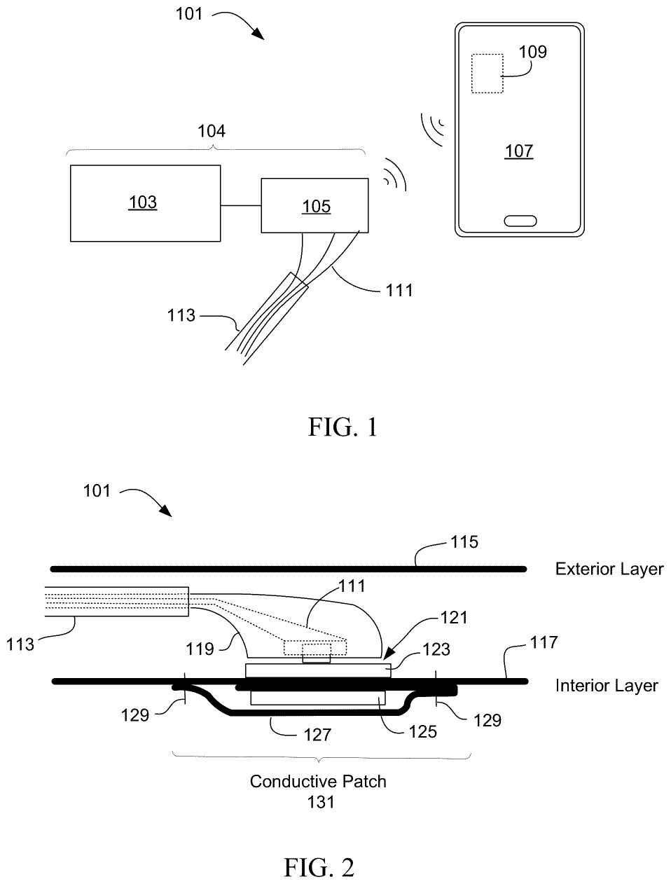

Referring now to FIGS. 1-2 in the drawings, a massage suit system is illustrated. As stated previously, system 101 includes a wearable garment that is configured to include a pair of electrodes configured to selectively provide electrical stimulation to a portion of a user's body. The wearable garment may be worn by a user in both static and dynamic motion environments. It is ideally suited to be worn by the user when the user is active, such as when walking, working, or doing other activities that require motion. System 101 is ideally suited to allow a user to receive e-stim treatment to selected portions of the body while in motion, or rather without having to remain in a resting position. It is understood that the wearable garment may be formed and structured to fit different parts of the body. Examples may include a shirt, shorts, a sleeve, a sock, and the like. The system is operable with one or more wearable garments.

The wearable garment includes an operating unit 104 configured to receive and transmit signals with a remote electronic device 107. Device 107 serves as a user interface to allow a user to regulate the performance of operating unit 104. Through device 107 a user may activate or deactivate one or more pairs of electrodes 119. Likewise device 107 may allow the user to regulate the performance of multiple wearable garments. Each wearable garment may be operable individually or in combination with any number of other wearable garments. Device 107 includes application software 109 configured to wirelessly communicate with operating unit 104.

As seen in particular with FIG. 2, it should be understood that the wearable garment may refer to a single layer of fabric or multiple layers of fabric. In FIG. 2 an interior layer 117 and an exterior layer 115 are illustrated. The wearable garment need only an interior layer 117, which would then serve as the only layer of fabric. However, system 101 can also include multiple layers wherein a secondary layer, exterior layer 115, may serve to conceal electrode 119 along with wires 111 from view. Sandwiching the wiring between layers provides some visual discretion and privacy to the user. For purposes herein, the wearable garment will include both layers 115 and 117. It should be stated that fabric used for the wearable garment maybe that of any type of fabric.

In particular with FIG. 1 of the drawings, electronic device 107 is shown in wireless communication with operating unit 104. Operating unit 104 is configured to include a power supply 103 and a control unit 105. Control unit 105 includes a communication suite to permit wireless communications. Additionally, control unit 105 is configured to process data received from device 107 and selectively send electrical current to a pair of electrodes 119. The electrical current may be supplied through power supply 103. It is understood that each wearable garment includes its own operating unit 104. Device 107 is operable with one or more wearable garments and is configured to operate each individually or in any numbered collective group as desired by the user. This permits a user the functionality to turn on or off a wearable garment for the arm while a wearable garment sock remains active, for example. The user is able to customize which electrodes in which wearable garments are on or off.

Device 107 further serves as a user interface to permit the user to customize different facets of system 101. For example, a user may turn on or off selected electrodes, turn on or off wearable garments, regulate the intensity and or intensity of electrical current to the electrodes, and the like. Other functionalities may include timers, tracking performance data, diagnosis capabilities, and so forth. Is also understood that operating unit 104 may be programmed with a set level of instructions such that if and when device 107 loses wireless communication with operating unit 104, operating 104 may continue without interruption. Control unit 105 may include one or more processors, input/output interfaces, and data storage devices to allow this functionality.

Control unit 105 is coupled to wires 111 which are routed between layers 115 and 117 of the wearable garment. A tube 113 is configured to pass around wires 111 so as to protect and group the wires together when routed. Tube 113 extends between control unit 105 and electrode 119. Electrode 119 is configured to selectively couple to a conductive patch 131. Patch 131 is coupled to a second side of interior layer 117 which is opposite a first side adjacent electrode 119. Electrical current that passes through wires 111 and to electrode 119 are dispersed through conductive patch 131 in contact with a user's body.

Conductive patch 131 includes a conductive fabric 127 and a connector 121. Connector 121 is configured to releasably coupled to electrode 119. Electrode 119 may be configured to magnetically couple to connector 121 to permit easier detachment. Connector 121 is also configured to pass electrical current from electrode 119 through layer 117 such that the electrical current contacts the user's body. One way in which this may be done is where connector 121 is a snap that includes 2 portions, namely portion 123 and portion 125. In this manner the snap would sandwich layer 117 and pass through layer 117 such that both portions are conductively coupled. It is understood that other methods may be used. It is important that connector 121 be capable of receiving electrical current from a first side of layer 117 and pass it to a second side of layer 117.

Customarily electrodes 119 are coupled to patches that are attached directly to the user's body. Such a configuration can lead to burns and discomfort on the users body. Therefore it is ideal that the electrical current be dispersed to a larger surface area on the body. This would allow for a wider range of intensity or strength in the electrical current. System 101 includes conductive fabric 127 that is coupled to connector 124 and is configured to receive the electrical current from electrode 119 and disperse it across the surface area of its fabric. The size and shape of conductive fabric 127 is dictated by design constraints and maybe any size necessary. To address the potential burning and discomfort a user may receive through e-stim treatments via electrode 119, electrical fabric 127 is selectively overlaid or overlaps connector 124. Connector 124 is configured to grasp or secure onto fabric 127 such that a conductive connection is realized. As seen in FIG. 2, connector 124 is shown sandwiching fabric 127 between portions 123 and 125. Conductive patch 131 is configured to overlay conductive fabric 127 onto itself so as to fully cover portion 125, or conductor 124, along the second side of layer 117. Conductive layer 127 is then stitched 129 or affixed two layer 117 around its periphery. The stitching around his periphery prevents conductive fabric 127 from moving and exposing connector 124.

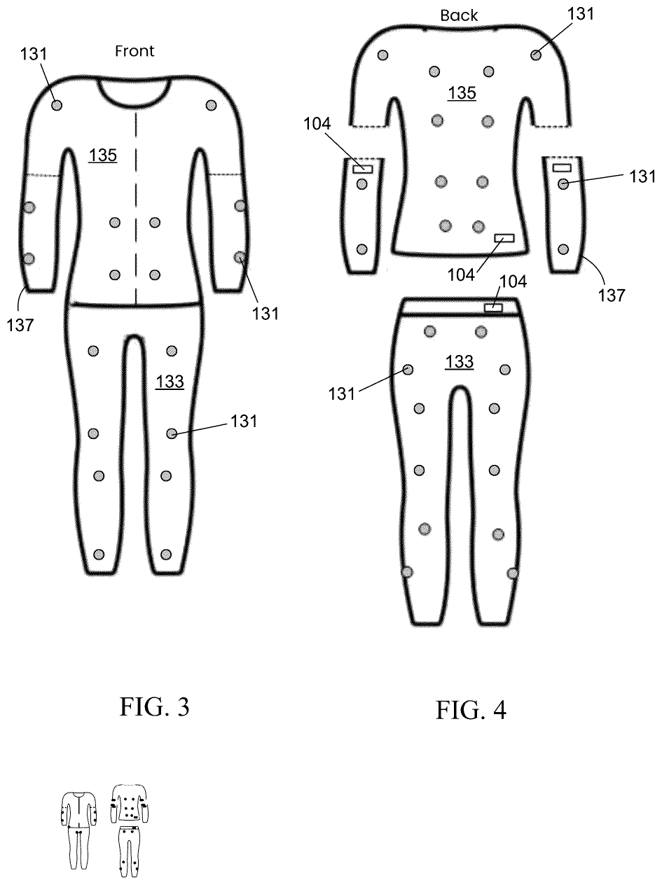

Referring now also to FIGS. 3-4 in the drawings, representative views of wearable garments are illustrated. In FIG. 3 a front view of exemplary wearable garments are provided while FIG. 4 illustrates a corresponding rear view of wearable garments. It is understood that FIG. 4 illustrates a partially exploded view such that different wearable garments are separated from each other. As noted previously, the wearable garment may be sized and shaped to fit any portion of a user's body. For example, sleeve 137 is shown capable of being detached from shirt 135. Pants 133 are also shown which may be separated from shirt 135. The garments illustrated in these Figures provide examples of different types of garments that may be used. It should be understood that this is not exhaustive, nor should it be meant to be limiting as other types of garments may be used by a user.

From these figures exemplary locations of conductive patches 131 are provided. Patches 131 may be distributed to any portion of the wearable garments. Ideal locations are those that are aligned with desired muscle groups. Also visible are operating units 104. Each wearable garment includes an individual operating unit 104 that is configured to regulate the electrodes on that wearable garment. Each operating unit is in wireless communication with device 107 through software application 109. More conductive patch areas may be located on the wearable garments than shown. There's merely illustrate representative locations.

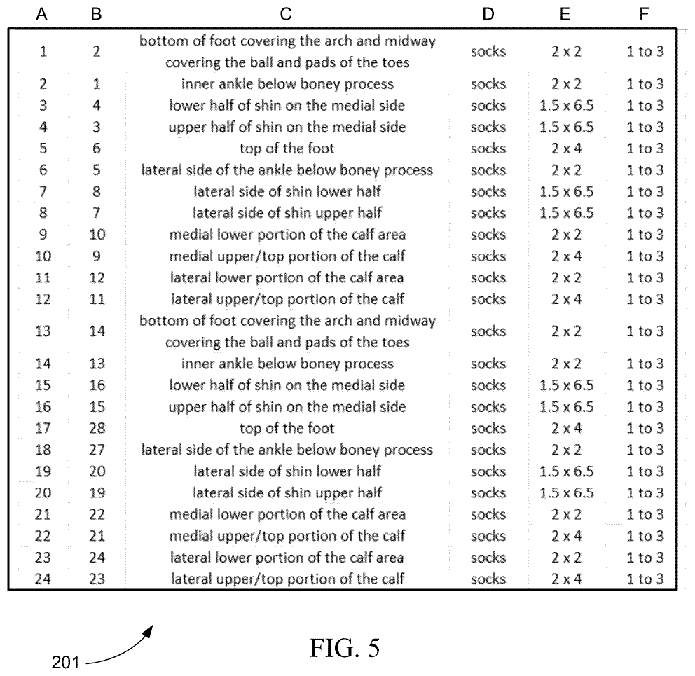

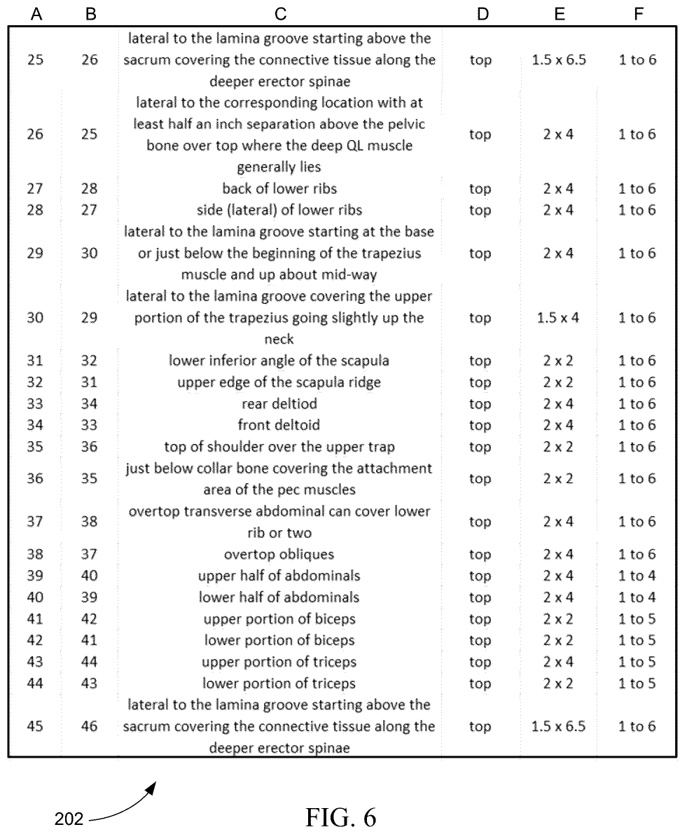

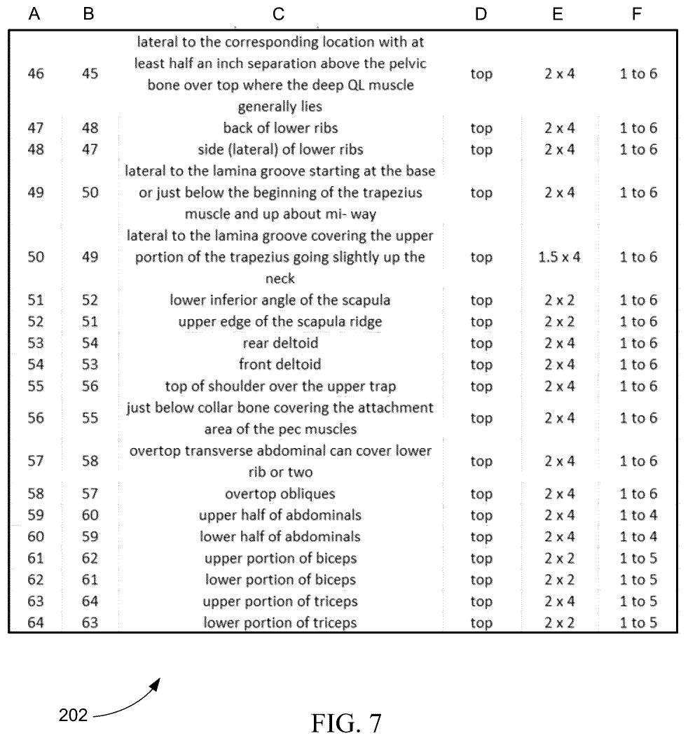

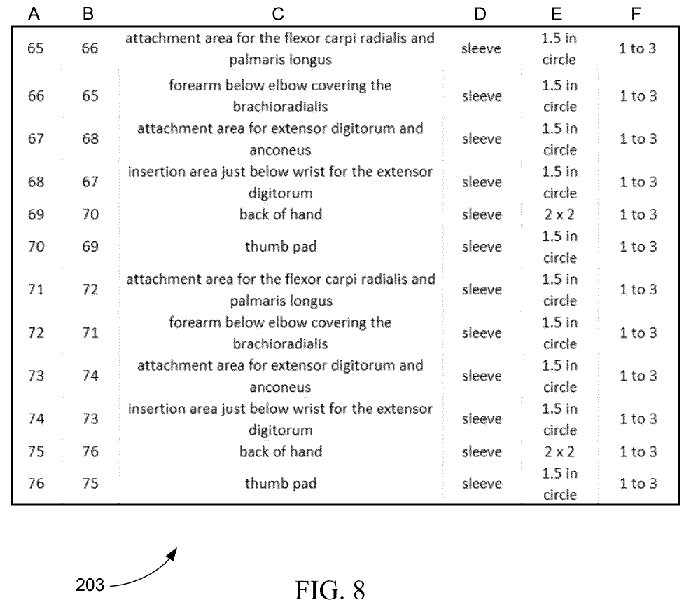

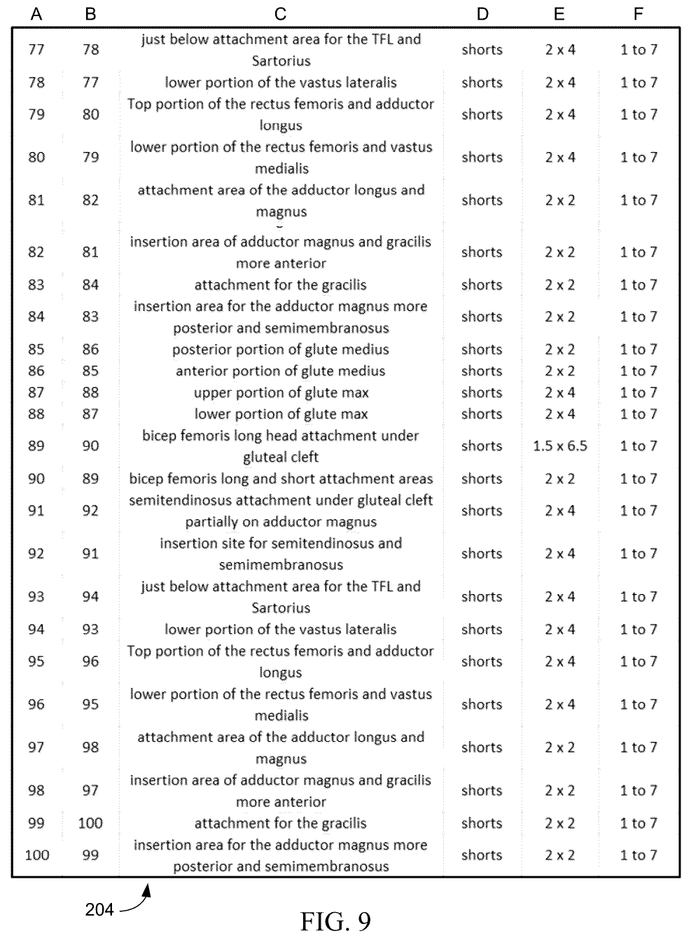

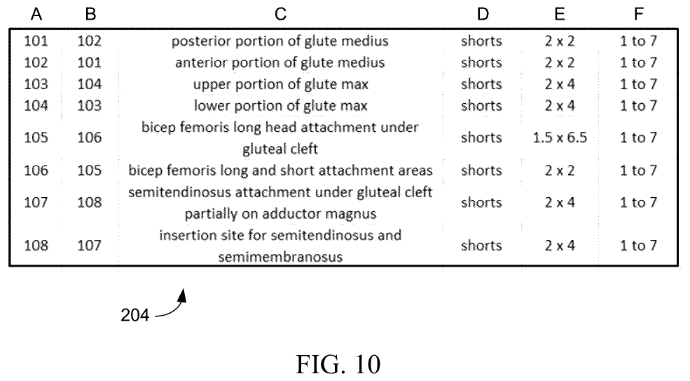

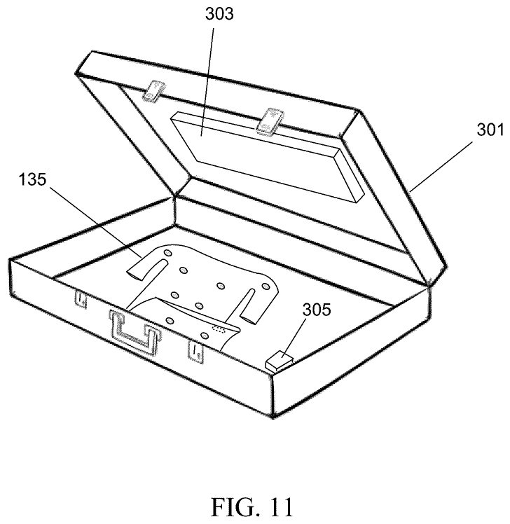

Referring now to FIGS. 5-10 in the drawings, e-stim tables are provided. Each table is configured to provide electrode information related to a particular wearable garment. For example, table 201 in FIG. 5 is for a sock garment. FIGS. 6-7 illustrate table 202 in relation to a top or shirt garment (similar to 135). FIG. 8 illustrates table 203 in relation to a sleeve garment (similar to 137). FIGS. 9-10 illustrate table 204 in relation to a shorts garment.

Each table provides columns of information labeled A through F. Column A relates to the electrode number. The numbering of electrodes may be arbitrary and chosen in any desired way. Column B relates to a corresponding electrode number. During E stim treatments an electrical current is passed through the body between 2 electrodes. In column A is the first electrode and in column B is the corresponding second electrode that correlates with the first electrode. Again, the numbering of particular electrodes is exemplary and is used to illustrate the corresponding characteristics between a pair of electrodes to address particular areas of the body. Column C is the location of the conductive patch 131 relative to the body. Column D illustrates the type of wearable garment. Column E is the relative exemplary pad size that contacts the body. As noted previously, conductive fabric 127 overlays connector 124 and is stitched or affixed to layer 117. The surface area of the exposed conductive fabric 127 is the pad size. It is understood that the pad size may affect the intensity or strength of the treatment. It is also understood that the pad size may be selected based upon the type of muscle or tissue being treated. Column F illustrates an intensity level rating for use with each electrode. The intensity rating is provided on a scale from 1-10 with 10 being the highest intensity. The amount of current or frequency capable of being provided through operating unit 104 may be scaled to be within a 1-10 intensity rating scale. Again, it is understood that the precise locations in Column C are not meant to be limiting. Other locations may be identified and targeted through system 101.

Referring now also to FIG. 11 in the drawings, a cleaning case 301 is illustrated. Wearable garments within system 101 will get dirty overtime. The wearable garments are configured to be cleaned but as they contain electrical components, conventional cleaning methods may not be advised. System 101 may include a portable cleaning case 301. Case 301 is configured to receive electrical power from one or more power sources, such as a rechargeable battery or an outlet. Case 101 includes a UV light 303 (ultraviolet light) within an interior volume of case 301. The UV light 303 is configured to shine upon wearable garments and to kill living organisms such as germs. One or more wearable garments may be located within case 301. One or more ultraviolet lights 303 may be provided. Lighting may be located on one or more surfaces along the interior of case 301. Another function of case 301 is to include a charging unit 305 configured to provide charging to operating unit 104. Charging may be performed through either a wired or wireless connection. One or more wearable garments may be charged simultaneously.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

What is claimed is:

1. A massage suit system, comprising:
a wearable garment;
an operating unit configured to include a power supply and a control unit, the operating unit coupled to a first side of the wearable garment;
a pair of electrodes in electrical communication with the control unit;
a pair of conductive patches each coupled with one of the pair of electrodes; and
an electronic device configured to communicate with the operating unit, the electronic device configured to send and receive data to the control unit, the control unit configured to regulate the transmission of electrical current to and from the pair of electrodes.

2. The system of claim 1, wherein the pair of conductive patches is configured to receive the electrical current from the pair of electrodes and disperse it over its surface area.

3. The system of claim 1, wherein the system is functional in both static and dynamic motion environments.

4. The system of claim 1, wherein the wearable garment is configured to be cleaned.

5. The system of claim 4, further comprising:
a portable case having a UV light within its interior volume, the UV light configured to clean the wearable garment.

6. The system of claim 1, wherein the pair of electrodes are magnetically coupled to the pair of conductive patches.

7. The system of claim 1, wherein the wearable garment includes an outer layer of fabric configured to overlay across the first side so as to conceal the operating unit and pair of electrodes.

8. The system of claim 1, wherein the wearable garment is adapted to conform to a portion of a user's body.

9. The system of claim 1, wherein one of the pair of conductive patches includes a conductive fabric and a connector, the conductive fabric located on a second side of the wearable garment, the connector configured to affix the location of the conductive fabric to the wearable garment.

10. The system of claim 9, wherein the connector is configured to convey electrical current from the electrode to the conductive fabric.

11. The system of claim 9, wherein the conductive fabric overlaps the connector and is coupled around a periphery of the conductive fabric to the second side of the wearable garment.

12. The system of claim 9, wherein the conductive fabric is configured to receive electrical current from the electrode through the connector and disperse the electrical current across its surface.

13. The system of claim 9, wherein the conductive fabric overlaps and conceals the connector along the second side of the wearable garment.

14. A massage suit system, comprising:
a first wearable garment including:
a first operating unit having a power supply and a control unit, the operating unit coupled to a first side of the first wearable garment;
a pair of electrodes in electrical communication with the control unit, the pair of electrodes on the first side of the first wearable garment; and
a pair of conductive patches each coupled with one of the pair of electrodes on the first wearable garment, the pair of conductive patches located on the second side of the first wearable garment, the pair of conductive patches configured to receive electrical current from the pair of electrodes and disperse it along the second side of the first wearable garment;
a second wearable garment including:
a second operating unit having a power supply and a control unit, the second operating unit coupled to a first side of the second wearable garment; and
a pair of electrodes in electrical communication with the control unit, the pair of electrodes on the first side of the second wearable garment; and
a pair of conductive patches each coupled with one of the pair of electrodes on the second wearable garment, the pair of conductive patches located on the second side of the second wearable garment, the pair of conductive patches configured to receive electrical current from the pair of electrodes and disperse it along the second side of the second wearable garment; and
an electronic device configured to communicate with the operating units of both wearable garments, the electronic device configured to send and receive data to each of the control units, each control unit configured to regulate the transmission of electrical current to and from the pair of electrodes on its wearable garment.

15. The system of claim 14, wherein the operating units of both wearable garments are active simultaneously.

16. The system of claim 14, wherein the operating units of both wearable garments operate independently of each other.

17. The system of claim 14, further comprising:
a portable case configured to include a UV light within its interior volume, the UV lights are configured to clean the wearable garments.

18. The system of claim 17, wherein both wearable garments are cleaned simultaneously.

19. The system of claim 14, wherein the pair of electrodes are magnetically coupled to the respective connector.

20. The system of claim 14, wherein the first wearable garment includes an outer layer of fabric configured to overlay across the first side so as to conceal the first operating unit and pair of electrodes.
